(19) United States
(12) Patent Application Publication
(10) Pub. No.: US 2019/0307181 A1
(43) Pub. Date: Oct. 10, 2019

(54) BRASSIERE INCORPORATING AN ELECTRONC HOT FLASH MITIGATION SYSTEM

(71) Applicants: Margaret Githuku, Mason, OH (US); David Githuku, Mason, OH (US); Cory Mack, Venice, CA (US)

(72) Inventors: Margaret Githuku, Mason, OH (US); David Githuku, Mason, OH (US); Cory Mack, Venice, CA (US)

(21) Appl. No.: 16/379,919

(22) Filed: Apr. 10, 2019

Related U.S. Application Data
(60) Provisional application No. 62/655,399, filed on Apr. 10, 2018.

(51) Int. Cl. A41C 3/00; A61F 7/00; A61B 5/00

(52) U.S. Cl. CPC A41C 3/0064; A61F 7/00; A61B 5/0024; A61B 5/6804; A61F 2007/0093; A61F 2007/0011; A61F 2007/0021; A61F 2007/0068; A61B 5/4836

(57) ABSTRACT

A brassiere incorporating a hot flash mitigation system for cooling the upper torso, chest and neck area of a wearer is disclosed. The brassiere incorporating a hot flash mitigation system provides for a brassiere and at least one electronic hot flash mitigation system cooperatively associated thereto. The electronic hot flash mitigation system provides at least one fluid source having a valve in fluid engagement with an outlet thereof, at least one duct disposed proximate to one of the at least two front panel sections and in fluid engagement with the outlet of the fluid source, and at least one nozzle disposed upon an end of the duct distal from the fluid source and in fluid communication with the at least one duct.

### FIELD OF THE INVENTION

The present disclosure relates to a garment that supports the female mammary glands and provides cooling to the upper torso, chest and neck area of the wearer. The present disclosure more particularly relates to a garment suitable for assisting in the relief of the hot flashes associated with menopause and perimenopause by expelling air to targeted areas of the body such as the neck, chest, arms, back, or other regions of the body proximate to the brassiere.

### BACKGROUND OF THE INVENTION

Menopause is the time in a woman's life when the function of the ovaries ceases and she can no longer become pregnant. The ovary (female gonad), is one of a pair of reproductive glands in women. They are located in the pelvis, one on each side of the uterus. Each ovary is about the size and shape of an almond. The ovaries produce eggs (ova) and female hormones such as estrogen. During each monthly menstrual cycle, an egg is released from one ovary. The egg travels from the ovary through a Fallopian tube to the uterus.

The ovaries are the main source of female hormones, which control the development of female body characteristics such as the breasts, body shape, and body hair. The hormones also regulate the menstrual cycle and pregnancy.

Menopause is defined as the state of an absence of menstrual periods for 12 months. The menopausal transition starts with varying menstrual cycle length and ends with the final menstrual period. Perimenopause is a term sometimes used and means “the time around menopause.” It is often used to refer to the menopausal transitional period. Perimenopause is sometimes used to explain certain aspects of the menopause transition in lay terms.

Menopause occurs because of the sharp decrease of estradiol and progesterone production by the ovaries. After menopause, estrogen continues to be produced mostly by aromatase in fat tissues and is produced in small amounts in many other tissues such as ovaries, bone, blood vessels, and the brain where it acts locally. The substantial fall in circulating estradiol levels at menopause impacts many tissues, from brain to skin. Additionally, in contrast to the sudden fall in estradiol during menopause, the levels of total and free testosterone, as well as dehydroepiandrosterone sulfate (DHEAS) and androstenedione appear to decline more or less steadily with age. An effect of natural menopause on circulating androgen levels has not been observed.

Before menopause, a woman's periods typically become irregular, which means that periods may be longer or shorter in duration or be lighter or heavier in the amount of flow. During this time, women often experience hot flashes and other vasomotor symptoms. A hot flash is a feeling of warmth that spreads over the body and is often most pronounced in the head and chest. A hot flash is sometimes associated with flushing and is sometimes followed by perspiration. These menopausal transition symptoms typically last from 30 seconds to ten minutes and may be associated with shivering, sweating, and reddening of the skin. Although the exact cause of hot flashes is not fully understood, hot flashes are likely due to a combination of hormonal and biochemical fluctuations brought on by declining estrogen levels.

There is currently no method to predict when hot flashes will begin and how long they will last. Hot flashes occur in up to 40% of regularly menstruating women in their forties, so they may begin before the menstrual irregularities characteristic of menopause even begin. About 80% of women will be finished having hot flashes after five years. Sometimes (in about 10% of women), hot flashes can last as long as 10 years. There is no way to predict when hot flashes will cease, though they tend to decrease in frequency over time. They may also wax and wane in their severity. The average woman who has hot flashes will have them for about five years.

The current management of hot flashes generally consist of managing the current lifestyle. Current hot flash mitigation management techniques only include measures such as drinking cold liquids, staying in cool rooms, using fans, removing excess clothing, and avoiding hot flash triggers such as hot drinks, spicy foods, etc. These techniques may even be partially supplemented by the use of medication.

A brassiere (or bra) is a form-fitting undergarment designed to support a woman's breasts. Mass-produced bras are manufactured to fit a prototypical woman standing with both arms at her sides.

A bra is typically a complicated garment to make. A common design can have between 20 and 48 component parts, including the band, hooks, cups, lining, and straps. The main components of a brassiere provide for a chest band that wraps around the torso, two cups attached thereto, and accompanying shoulder straps. The chest band is usually closable in the back by the use of a hook and eye fastener. However, the chest band may also be fastened at the front. Sleep bras or athletic bras do not have fasteners and are pulled on over the head and breasts. The section between the cups is called a gore. The section under the armpit where the band joins the cups is called the “back wing”.

Bra components, including the cup top and bottom (if seamed), the central, side and back panels, and straps, are cut to manufacturer's specifications. Many layers of fabric may be cut at the same time using computer-controlled lasers or bandsaw shearing devices. The pieces are assembled by piece workers using industrial sewing machines or automated machines. Coated metal hooks and eyes are sewn in by machine and heat processed or ironed into the back ends of the band and a tag or label is attached or printed onto the bra itself.

The chest band and cups of a typical brassiere, not the shoulder straps, are designed to support the weight of women's breasts. Strapless bras rely on an underwire and additional seaming and stiffening panels to support them. The shoulder straps of some sports bras cross over at the back to take the pressure off the shoulders when arms are raised. In short, a brassiere is intended to physically maintain the shape of the bust in a manner that intends to maintain, and not alter, the original or biologically presented bust or breast material.

As shown in FIGS. 1 and 2, an exemplary prior art brassiere (bra) 100 (greatly simplified from the construction detailed above solely for reasons of brevity) can generally comprise two mirror-image front panel sections 120 each in the general form of a cup (and known to those of skill in the art as a cup(s)) and at least two rear panel sections 110 (known to those of skill in the art as strap(s)). In some embodiments the at least two rear panel sections 110 can be cooperatively attached in mating engagement with each other with a clip, fastener, hook and loops, and the like so that the brassiere 100 may be easily placed (by placing the ends of each rear panel section in connective engagement) and/or removed (by disassociating the ends of each rear panel section from connective engagement). In other embodiments the two rear panel sections 110 may be cooperatively attached via a sewn seam or other form of fixable attachment known to those of skill in the art. In other embodiments, a single rear panel made of elastic material(s) can extend from a portion of one front panel section 120 to a portion of the other front panel section 120 and is meant to be worn across the back of the wearer.

As shown in the exemplary front elevational view of FIG. 2, the two front panels 120 may be connected via a connecting member 130. This member may be a seam, elastic material, a strap, or simply the region that connects the left and right 120 two front panels 120.

As shown in FIGS. 1 and 2, exemplary embodiments of the prior art may incorporate a respective shoulder strap 140 that extends from a first position disposed upon a respective front panel 120 over the shoulder of the wearer and cooperatively interfacing with a respective rear panel section 110 cooperatively associated with the respective front panel 120. The shoulder strap may interface with the respective front panel 120 and respective rear panel section 110 via a stitch or seam or via a fastener such as a grommet or loop. For increased comfort of the wearer, the shoulder strap 140 may be adjustable in length, toughness, or angle.

One of skill in the art will readily appreciate that there are no currently known devices that can be incorporated or are presently incorporatable with existing brassiere technology that would be useful to mitigate the symptoms associated with a menopausal hot flash. Therefore, there is a need for, and a clear victory for, a brassiere that could provide at least partial relief from menopausal hot flashes by the use of a motive fluid (e.g., air) and/or an invasive fluid (e.g., air). Additionally, there is a clear need for a menopausal hot flash mitigation system for incorporation into existing brassiere constructions and technology to provide at least partial relief from menopausal hot flashes.

### SUMMARY OF THE INVENTION

The present disclosure provides for a brassiere incorporating a hot flash mitigation system for cooling the upper torso, chest and neck area of a wearer. The brassiere comprises a brassiere and at least one electronic hot flash mitigation system cooperatively associated thereto. The brassiere comprises two front panel sections connectively engaged to each other and at least one rear panel section connectively engaged to each of the two front panel sections. The connectively engaged two front panel sections and the at least one rear panel section form a wrap capable of encircling the wearer or intended wearer's body. The electronic hot flash mitigation system (eHFMS) comprises at least one fluid source having a valve in fluid engagement with an outlet thereof, at least one duct disposed proximate to one of the at least two front panel sections and in fluid engagement with the outlet of the fluid source, and at least one nozzle disposed upon an end of the duct distal from the fluid source and in fluid communication with the at least one duct. The valve has a controller cooperatively associated thereto. The controller operates and motivates the valve from a first position wherein a fluid contained in the fluid source cannot egress from the fluid source to a second position wherein the fluid contained within the fluid source can egress from the fluid source to an outlet in cooperative fluid engagement thereto. The at least one nozzle operatively conveys the fluid from the duct to the upper torso, chest and neck area of the wearer.

### BRIEF DESCRIPTION OF THE DRAWINGS

FIG. 1 is a front ¾ elevational view of an exemplary prior art brassiere;

FIG. 2 is a front elevational view of the exemplary prior art brassiere of FIG. 1;

FIG. 3 is a front ¾ elevational view of an exemplary brassiere incorporating the electronic hot flash mitigation system of the present disclosure;

FIG. 4 is a front elevational view of the exemplary brassiere incorporating the electronic hot flash mitigation system of FIG. 4;

FIG. 5 is a plan view of an exemplary electronic hot flash mitigation system;

FIG. 5A is a plan view of an alternative electronic hot flash mitigation system;

FIG. 5B is a plan view of another alternative electronic hot flash mitigation system;

FIG. 5C is a plan view of yet another alternative electronic hot flash mitigation system;

FIG. 6 is a front ¾ elevational view of an exemplary brassiere incorporating the electronic hot flash mitigation system of the present disclosure further incorporating at least one rear panel section with cooperatively associated ducts;

FIG. 7 is a front elevational view of an exemplary brassiere incorporating the electronic hot flash mitigation system of FIG. 4 where certain ducts within the brassiere provide hot flash mitigation to the torso of the wearer;

FIG. 8 is a front ¾ elevational view of an exemplary garment incorporating the electronic hot flash mitigation system of the present disclosure; and,

FIG. 9 is a front elevational view of an exemplary brassiere incorporating the electronic hot flash mitigation system and a wireless body sensor network in communication with a smart device.

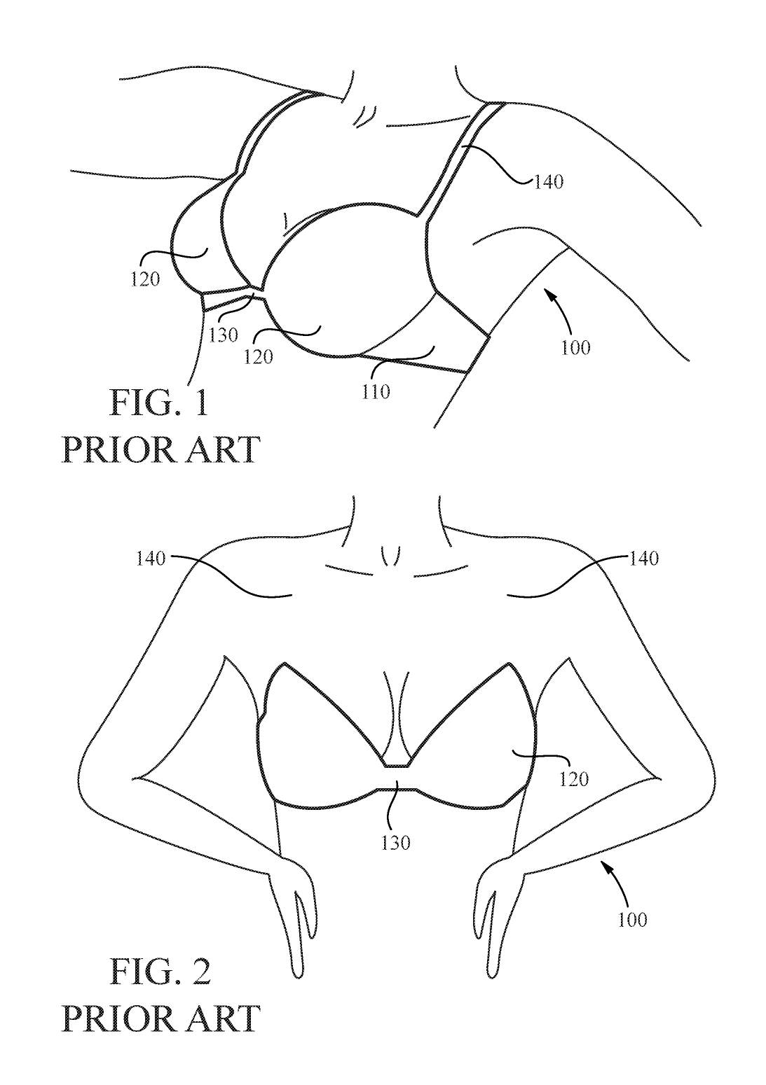

### DETAILED DESCRIPTION OF THE INVENTION

As used herein, a “fluid” is a substance that continually deforms (flows) under an applied shear stress. Fluids are a subset of the phases of matter and include liquids, gases, plasmas, and to some extent, plastic solids and flowable solids. A typical fluid suitable for use with the present disclosure is air. When used herein, the term “air” is merely illustrative and is intended to be used only as a non-limiting example of a fluid. In other words, “air” is intended to be an exemplary and non-limiting embodiment of a “fluid”. One of skill in the art would also understand that a fluid suitable for use with the present disclosure could also be considered a motive fluid (e.g., air) and/or an invasive fluid (e.g., air). One of skill in the art will also understand that a fluid may also have functional additives provided therein. Such functional additives could include perfumes, medicaments, therapeutic agents, combinations thereof and the like.

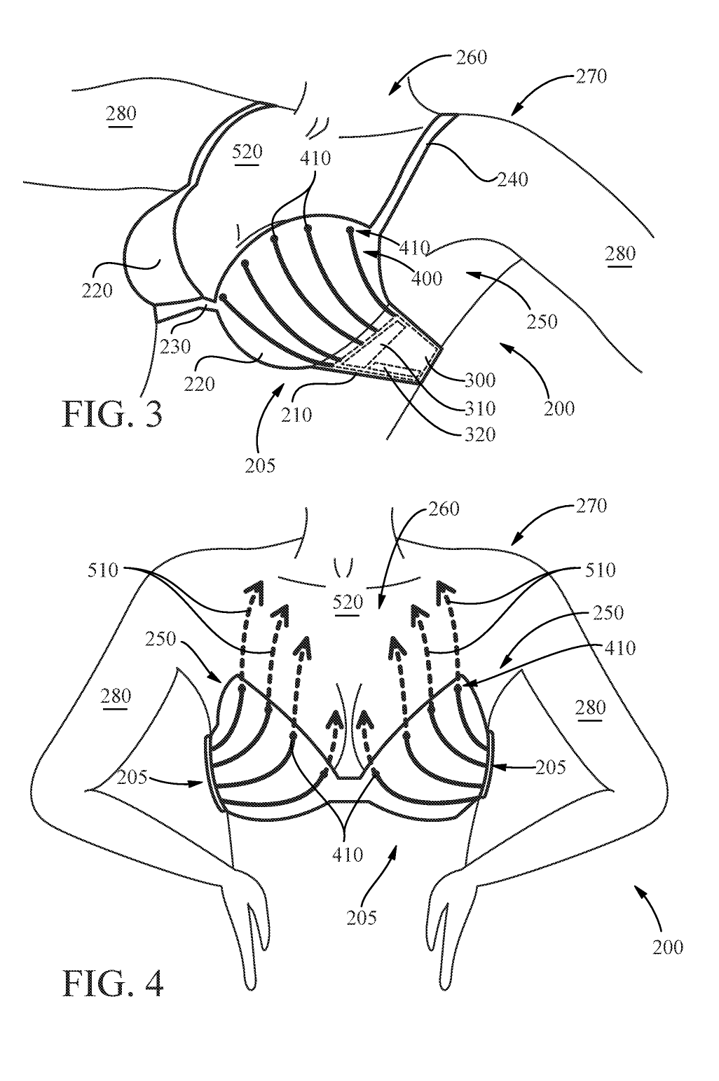

FIGS. 3 and 4 of the present disclosure depict an exemplary and non-limiting brassiere 200 incorporating the electronic hot flash mitigation system 205. As indicated supra, a brassiere is typically an assemblage of a plurality of parts. However, for the sake of brevity, the brassiere 200 of the present disclosure will be generally assembled from a few individual components.

One of skill in the art will recognize that the exact construction of the brassiere 200 is not a critical element of the present disclosure. Thus, the construction of the brassiere 200 can follow any design norm useful for providing a brassiere 200. In other words, these generally described individual components may in reality be a complex assembly of a plurality of components as indicated supra. On the other hand, for purposes of simplicity, the components of a brassiere 200 may be comprised of a few parts derived and/or designed as combinations or sub-combinations of the aforementioned brassiere components. In any regard the brassiere 200 of the present disclosure may be considered, constructed, and described in any manner consistent with either an assembly of a plurality of completely integrated components, a simpler assembly of components (described infra), or a purposeful combination of components required to produce a brassiere as would be understood by one of skill in the art. The process and complexity of brassiere construction should not detract from the present description.

Generally, a brassiere 200 of the present disclosure generally comprises two front panel sections 200 (also known to those of skill in the art as cups 200), at least one rear panel section 210 (also known to those of skill in the art as a strap 210), and optional shoulder straps 240. A top hole 260 provides for placement of the brassiere 200 over the head and over the shoulders 270 of the wearer. A pair of opposed arm holes 250 each disposed upon opposed sides of the brassiere 200 can provide for the insertion of the arms 280 therethrough. Here, portions of fabric disposed between the top hole 260 and each of the arm holes 250 can capably form the optional shoulder straps 240 that can properly overlay the wearer's shoulders 270. However, referring to FIG. 4, one of skill in the art will recognize that shoulder straps 240 are not completely necessary. In some constructions, it may be desirable that the two front panel sections 200 can only be connectively engaged to the at least one rear panel section 210 (also referred to herein as back panel 225) to form a de facto wrap that encircles the wearer or intended wearer's body without the need for shoulder straps 240.

The two front panels 220 may be fixably and permanently connected via a connecting member 230. This member may be a seam, an elastic material, a strap, or simply a region of connection between the mirror image left and right two front panels 220. Alternatively, the two front panels 220 may be releasably connected to each other at opposing edges for securing the two front panels 220 together when the brassiere 200 is worn.

Additionally, the at least one rear panel section 210 can be provided as a plurality of connectable segments. In a preferred embodiment, a first of the connectable segments (or sections) is fixably attached to one of the two front panels 220 and a second connectable section being fixably attached to the other of the two front panel sections 220. Each of the plurality of connectable sections can be cooperatively attached in mating engagement to each other at an end distal from the fixable connection to the respective panel section of the two front panels 220 with a clip, fastener, hook and loops, and the like, so that the brassiere 200 may be easily placed (by placing the ends of each rear panel section in connective engagement) and/or removed (by disassociating the ends of each rear panel section from connective engagement).

In some embodiments, two rear panel sections 210 are attached with a clip or fastener so that it may be easily removed. In other embodiments, two rear sections 210 may be attached via a seam, and in others still there is one rear panel section 210 made of an elastic material that can extends from one front panel section 220 to the other front panel section 220 and is meant to be worn across the back of the wearer. In other embodiments, two rear panel sections forming the at least one rear panel section 210 may be cooperatively attached via a sewn seam or other form of fixable attachment known to those of skill in the art. In other embodiments, a single rear panel section 210 made of an elastic material(s) can extend from a portion of one front panel section 220 to a portion of the other front panel section 220 (forming a loop) and is meant to be worn across the back of the wearer. It is believed that other methods of facilitating the securement of the brassiere 200 to the wearer are feasible for use and can be successfully incorporated with the brassiere 200 envisioned for use herein.

As mentioned supra, a shoulder strap 240 can extend from a respective panel section of the two front panel sections 220 over the wearer's shoulders 270 and interfacing with the at least one rear panel section 210. The shoulder strap may interface and connectively engage with a respective panel section of the two front panel sections 220 and the at least one rear panel section 210 via a stitch, seam, or fastener such as a grommet or loop. For increased comfort of the wearer, the shoulder strap 240 may be adjustable in length, toughness, or angle.

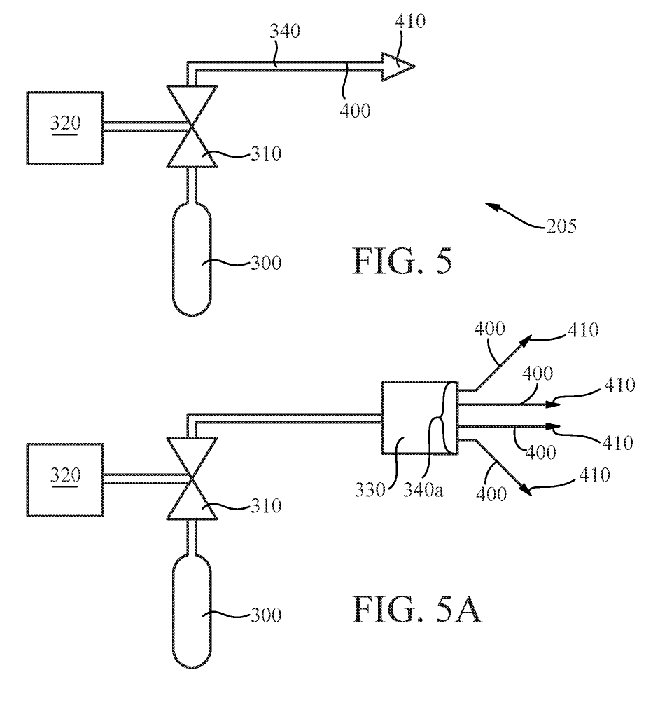

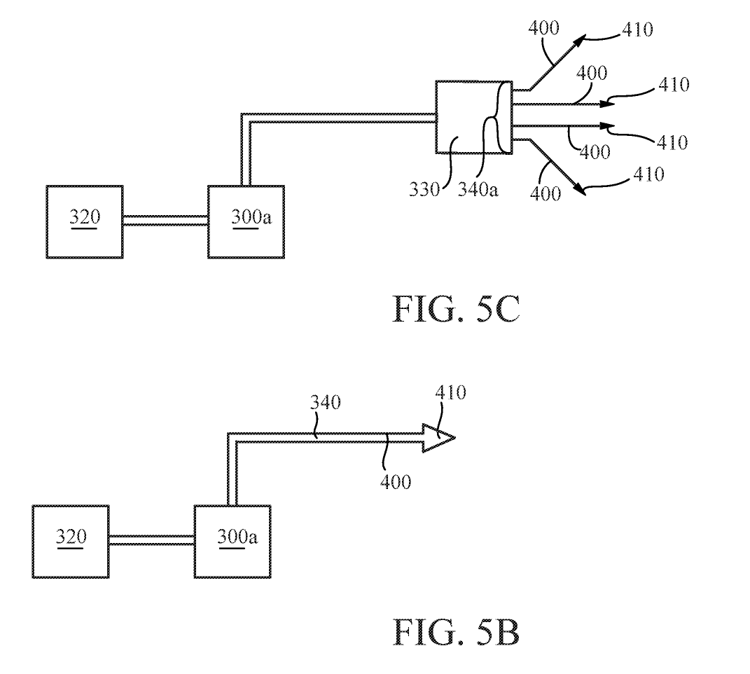

FIGS. 5-5C depict exemplary but non-limiting embodiments of an electronic hot flash mitigation systems (eHFMS) 205 that can be suitably disposed in cooperative, mating, contacting, and fluid engagement (i.e., integral with) with a brassiere 200. Alternatively, one of skill in the art will readily recognize that eHFMS 205, or even components thereof, can be disposed in communicating engagement with brassiere 200 but not integral with brassiere 200. For example, the complete eHFMS 205 may be disposed in a shirt pocket of a wearer but remain in fluid engagement with brassiere 200. Alternatively, a portion of the eHFMS 205 may be disposed in the shirt pocket of a wearer but remain in communicating engagement with the remainder of eHFMS 205 while that remainder of the eHFMS 205 is disposed integral with, and in fluid communication with, brassiere 200. Several exemplary but non-limiting embodiments are presented infra.

In function, the eHFMS 205 incorporates at least one fluid source 300, an optional valve 310, and a controller 320. The fluid source can be provided in several alternative manners.

First, as shown in FIGS. 5 and 5A, an exemplary but non-limiting fluid source 300 can be a pressurized fluid containing vessel (e.g., a source to deliver a pressure head) such as a CO₂ cartridge typically found commercially as a saleable, replaceable product. For example, a typically purchased CO₂ cartridge is a small metal container, about the size of a thumb, that can hold highly pressurized CO₂ (carbon dioxide) gas. One of skill in the art will understand that at 21.1° C., CO₂ obtains a gas pressure of 58.0 atm when confined in a vessel. If there is more CO₂ in placed in the vessel, it will exist in liquid form. A suitable device for fluid source 300 is a 12 gram CO₂ cartridge, known commercially as a Powerlet , can contain about 8 grams of CO₂ in a liquid/gas equilibrium. The cartridge is made of thick steel with a thin welded cap. A suitable source of commercially available pressurized fluid containing vessel suitable for use as fluid source 300 is Soda Plus 8 Gram CO₂ Cartridges 10 Pack available from Gas Depot, Fort Lauderdale, Fla. It should be understood that a fluid source 300 may be self-contained and/or replaceable. In any regard, a suitable fluid source 300 can be cooperatively integratable with eHFMS 205.

Alternatively, an exemplary but non-limiting fluid source 300 can be a ‘bag-on-valve’ technology pressurized fluid containing also found commercially as a saleable, replaceable product. Bag-on-valve technology consists of an aerosol valve operatively and fluidly connected to a welded bag disposed within a container. The product to be dispensed is placed inside the bag while a propellant, such as compressed air/nitrogen, is filled under pressure in the space between bag and container. The product is dispensed by the propellant that simply squeezes the bag when the spray button is pressed. The product is then expelled from the bag by the propellant. The product can then be fluidly conducted as required by the eHFMS 205. Such an execution can always maintain product integrity by remaining separated from the propellant.

Second, as shown in FIGS. 5B and 5C, an exemplary but non-limiting fluid source 300A could be provided as a simple mechanical fan and/or micro-fan. A fan is a powered machine used to create flow within a fluid. A fan consists of a rotating arrangement of vanes or blades which act on the air. The rotating assembly of blades and hub is known as an impeller, a rotor, or a runner. Usually, it is contained within a housing or case. One of skill in the art will understand that mechanically, a fan can be any revolving vane or vanes used for producing currents of air. A fan can produce air flows with high volume and low pressure.

Third, as shown in FIGS. 5B and 5C, an exemplary but non-limiting fluid source 300A can be provided as an electrically driven mechanical pump and/or micro-pump. Several examples of mechanical pumps can include reciprocating pumps, diaphragm pumps, and double-acting pumps. Reciprocating pumps have a piston inside and two one-way valves—one going to the object and one to the outside air. When the pump is pulled up, the pressure inside decreases. This pulls in air from the outside but closes the valve to the object. When the piston is pushed down again, it compresses the air inside. This closes the inlet valve and opens the valve to the object.

A diaphragm pump has a flexible diaphragm. When an external force is applied, the diaphragm compresses, forcing air into whatever it is being pumped into. When the force is removed, the springy diaphragm expands again, drawing more air in from outside.

Double-acting pumps use two or more cylinders. When the plunger is pushed down on a double-acting pump, it compresses one cylinder, pushing air out. At the same time, the other cylinder is pulled open, drawing in air from the outside. When the plunger is pulled back up, the open cylinder is compressed, and the compressed cylinder is pulled up.

One of skill in the art would understand that a “closed-loop” system employs a pumped fluid that circulates in a closed loop without any exposure to the local environment and typically without the transfer of fluid into or out of the closed loop. One of skill in the art would understand that the present device incorporating the eHFMS 205 provides an “open-loop” system. One of skill in the art would understand that an open-loop system provides for the pumped fluid to be exposed to the local atmosphere at some point in the circuit.

Any valve suitable for controlling and/or regulating the flow of fluid from fluid source 300 is suitable for use with eHFMS 205. An exemplary valve 310 suitable for use with eHFMS 205 and suitable to regulate the output of fluid source 300 is an electrical control valve. The opening or closing of valve 310 can be done by an electrical actuator. Normally with a modulating valve, which can be set to any position between fully open and fully closed, valve positioners are used to ensure the valve attains the desired degree of opening. An electrically-operated valve 310 can require cabling and switch gear to activate.

An electrically-operated valve 310 provided as an automatic control valve generally consists of three main parts: a valve actuator, a valve positioner, and a valve body. The valve actuator moves the valve's modulating element, such as ball or butterfly. The valve positioner ensures the valve has reached the desired degree of opening. This overcomes the problems of friction and wear. The valve body provides containment of the modulating element such as a plug, globe, ball or butterfly. All components of a valve suitable for use as valve 310 can exist in several types and designs. A solenoid may be incorporated into the valve 310 to assist with operating the modulating element.

Further, electrical control of the valve 310 can also include a “smart” communication signal superimposed on the control current so that the health and verification of the valve position can be signaled back to a controller. The HART, Fieldbus Foundation, and Profibus are the most common control signal protocols. Suitable controllers can be wireless remote keys, smart devices, computers, and the like.

As shown in the non-limiting embodiment of FIGS. 5 and 5A, the controller 320 can operate the valve 310 by causing the valve 310 to activate and motivate from a first position wherein a fluid contained in the fluid source 300 cannot egress from the fluid source 300 to a second position wherein the fluid contained within the fluid source 300 can egress from the fluid source 300. The fluid egressing from the fluid source 300 is preferably directed toward an outlet 310 in cooperative fluid engagement with the outlet of the valve 310. When a necessary and sufficient quantity of fluid is egressed from the fluid source 300 to perform the requisite purpose, the controller 320 preferably operates, activates, and motivates the valve 310 from the second position wherein the fluid contained within the fluid source 300 can egress from the fluid source 300 back to the first position where the fluid contained in the fluid source 300 cannot egress from the fluid source 300.

Alternatively, and as shown in FIGS. 5B and 5C, the controller 320 can directly operate the the fluid source 300A to facilitate the egress of fluid from the fluid source 300A by turning the fluid source 300A on (e.g., fluid source 300A is provided as a mechanical fan) to a second position where the fluid source 300A is turned off and no fluid can egress from the fluid source 300A. The fluid egressing from the fluid source 300A is preferably directed toward an outlet 340 (FIG. 5B) or a plenum 330 having an outlet 340A in cooperative fluid engagement with the output of fluid source 300A. When a necessary and sufficient quantity of fluid has egressed from the fluid source 300A to perform the requisite purpose, the controller 320 can cease the operation, activation, and/or motivation of the fluid source 300A where fluid cannot egress from the fluid source 300A.

An exemplary but non-limiting controller 320 suitable for eHFMS 205 and integratable with valve 310 is a mechanical switch that is operated with direct wearer contact (i.e., the human is the controller). A mechanical switch (such as a push-button switch) provided as controller 320 can operate, activate, and/or motivate valve 310 by energizing a traditional power supply as would be understood by one of skill in the art to provide electrical energy to valve 310 thereby activating valve 310 to facilitate the egress of fluid from fluid source 300. The cessation of pressure to the mechanical switch can de-activate valve 310 resulting in the stoppage of fluid from fluid source 300.

Another exemplary but non-limiting controller 320 suitable for eHFMS 205 and integratable with valve 310 is a microcontroller. A microcontroller contains one or more CPUs (processor cores) along with memory and programmable input/output peripherals. Program memory in the form of ferroelectric RAM, NOR flash or OTP ROM is also often included on chip, as well as a small amount of RAM. A suitable microcontroller can be designed for an embedded applications such as the control of valve 310.

A suitable microcontroller can use four-bit words and operate at frequencies as low as 4 kHz, for low power consumption (e.g., single-digit milliwatts or microwatts). A suitable microcontroller can have the ability to retain functionality while waiting for an event such as a button press or other interrupt. Power consumption while sleeping (CPU clock and most peripherals off) may be just nanowatts, making many of microcontrollers well-suited for long lasting battery applications.

A microcontroller circuit incorporating a microcontroller suitable for use with eHFMS 205 can be operably coupled to and activated and/or deactivated with a remote control device. A suitable remote control devices can generally comprise a short-range radio transmitter and must be disposed within a certain range, usually 5-20 meters, of eHFMS 205. When a button is pushed, The remote control device can send a coded signal by radio waves to a receiver unit in the eHFMS 205, which activates or deactivates the eHFMS 205.

Alternatively, a microcontroller circuit incorporating a microcontroller suitable for use with eHFMS 205 can be operably coupled to and activated and/or deactivated with a Bluetooth network device. One of skill in the art will recognize that Bluetooth networking transmits data via low-power radio waves on a frequency from between about 2.402 GHz and about 2.480 GHz.

Bluetooth hardware comprises generally two parts - a radio device, responsible for modulating and transmitting the signal and a digital controller. The digital controller is likely a CPU, one of whose functions is to run a link controller and interfaces with the eHFMS 205. The link controller is responsible for the processing of the baseband and the management of ARQ and physical layer FEC protocols. In addition, it handles the transfer functions (both asynchronous and synchronous), audio coding and data encryption. The CPU of the device is responsible for attending the instructions related to Bluetooth of the host device, in order to simplify its operation. To do this, the CPU runs software called Link Manager that has the function of communicating with other devices through the LMP protocol.

A link manager establishes the communicating connection between the Bluetooth device and the eHFMS 205. The Bluetooth device can activate valve 310 as would be understood by one of skill in the art to activate valve 310 to facilitate the egress of fluid from fluid source 300. A stop signal from a Bluetooth device can accordingly cause the cessation of fluid from fluid source 300.

Alternatively, a microcontroller suitable for a controller 320 circuit incorporating a microcontroller 320 suitable for use with eHFMS 205 can be operably coupled to and activated and/or deactivated with a smart device. A smart device is an electronic device, generally connected to other devices or networks via different wireless protocols such as Bluetooth, NFC, Wi-Fi, LiFi, 3G, and the like that can operate to some extent interactively and autonomously. One type of smart devices is a smartphone. The term can also refer to a device that exhibits some properties of ubiquitous computing, including—although not necessarily—artificial intelligence. Smart devices can be designed to support a variety of form factors, a range of properties pertaining to ubiquitous computing and to be used in three main system environments: physical world, human-centered environments and distributed computing environments. A smart device, as the name suggests, is an electronic gadget that can connect, share and interact with its user and other smart devices through the so-called “internet of things.”. Although usually small in size, smart devices typically have the computing power of a few gigabytes. Smart devices are interactive electronic gadgets that understand simple commands sent by users and help in daily activities. Some of the most commonly used smart devices are smartphones, tablets, phablets, smartwatches, smart glasses and other personal electronics. While many smart devices are small, portable personal electronics, they are in fact defined by their ability to connect to a network to share and interact remotely.

Further, controller 320 can provide for voice activation. Voice activation or voice control allows the wearer to activate or control operation of the eHFMS 205 by simply using their voice, as opposed to pressing buttons to using a touchscreen interface device. For example, exemplary voice activation could be provided by an Amazon® Alexa® Voice Service (with devices such as the Amazon® Echo® and Amazon®Dot®), Google Home®, and Josh.ai.

One of skill in the art will understand that both Amazon® Alexa® and Google® Home® are essentially smart microphone devices. These are hardware platforms that can do language processing using a software layer. Josh is a full control system using a black box hidden in a closet or room which does all the language processing as well.

The voice activation process converts the spoken word into written text. Converting to the spoken word to written text using open-source ASR (Automatic Speech Recognition) technology.

It should also be understood that all electronics and electrical devices provided for herein, as well as any electrically-energized components can be powered by a self-contained power source (e.g., battery/batteries). Alternatively, any power required by electrical devices herein can be powered by a renewable resource (e.g., solar power).

Additionally, a wireless body sensor network can be incorporated with eHFMS 205. A smart device can be provided in communicative engagement with eHFMS 205 and the wireless body sensor network. Upon the occurrence of a biological trigger detected by the body sensor network, the smart device can provide an activation or deactivation signal to eHFMS 205 to activate or deactivate the flow of fluid from fluid source 300. The incorporation of a wireless body sensor network incorporated with eHFMS 205 is discussed infra.

Returning to FIGS. 3 and 4, an electronic hot flash mitigation system (eHFMS) 205 can be disposed in mating and contacting engagement with brassiere 200. In a preferred embodiment, an eHFMS 205 can be disposed proximate to each respective arm hole 250 and proximate to the area of connecting engagement between a front panel section 200 and a corresponding rear panel section 210 (i.e., the back wing).

As shown in FIGS. 5-5C, the outlet 340 of eHFMS 205 may be an opening or a slit(s). In the embodiment shown in FIGS. 5A and 5C, outlet 340A may be a region that provides a plenum 330, may be in communicating engagement with a plenum 330, and may also have the function of a one-way valve in that the pressurized fluid disposed and/or contained within fluid source 300 can exit fluid source 300 through outlet 340 when valve 310 is opened. The outlet 340 may be directly connected and in fluid engagement with a duct 400 as shown in FIGS. 5 and 5B. In FIGS. 5A and 5C, the outlet 340A may be cooperatively associated with a plenum 330 which is then connected and in fluid engagement with a plurality of ducts 400. Alternatively, plenum 330 may be integral with valve 310 and duct (ducts) 400 are in fluid communication with plenum 330.

As shown in FIGS. 3-5C, the eHFMS 205 can provide for at least one or more ducts 400 may each be fluidly connected to (i.e., in fluid engagement with) the outlet 340. By way of non-limiting example, a plurality of ducts 400 are each connected to outlet 340. Each duct 400 of the eHFMS 205 can be provided with a nozzle 410. It is believed that the presence of multiple ducts 400 can provide increased comfort for the relief of a hot flash as the fluid (e.g. air) conveyed from outlet 340 to nozzle 410 through duct 400 can be directed to multiple parts of the body such as upper torso, chest and neck area 520 of the wearer (this flow is shown by the arrows 510) as desired.

Additionally, nozzle 410 can comprise an educator-type nozzle to maximize fluid flow to upper torso, chest and neck area 520 of the wearer. One of skill in the art will recognize that an educator-type nozzle comprises a motive fluid inlet, an entrained fluid inlet, a diffuser, and an outlet. A motive fluid could be provided by fluid source 300. An entrained fluid is typically provided from the local environment proximate to eHFMS 205. The fluid exiting the educator-type nozzle is then directed toward the upper torso, chest and neck area 520 of the wearer.

Without desiring to be bound by theory, it is believed that one of skill in the art would understand that the increased comfort provided by the brassiere 200 from the fluid (e.g. air) expelled from the nozzle 410 toward the upper torso, chest and neck area 520 of the wearer can be attributable to the Joule-Thomson effect. In thermodynamics, the Joule-Thomson effect describes the temperature change of a real gas or liquid when it is forced through a valve while keeping the valve insulated so that no heat is exchanged with the environment. At room temperature, a fluid (e.g., air) expelled from a nozzle cools upon adiabatic expansion by the Joule-Thomson process.

In practice, the Joule-Thomson effect is achieved by allowing a gas to expand through a throttling device (such as the nozzle 410 described supra). No external work is extracted from the gas during this expansion. The cooling produced from this expansion is suitable for use in cooling processes.

As would be understood by one of skill in the art, two factors can change the temperature of a fluid during adiabatic expansion: 1. a change in internal energy of the fluid or 2. the conversion of the fluid's potential energy to kinetic internal energy. It is understood that temperature is the measure of thermal kinetic energy (i.e., the energy associated with molecular motion). Thus, a change in temperature of a fluid indicates a change in thermal kinetic energy. Since the internal energy of the fluid is the sum of the thermal kinetic energy and the thermal potential energy, even if the internal energy does not change, the temperature can change due to conversion between kinetic and potential energy. In short, an adiabatic free expansion typically produces a decrease in temperature of the fluid as it expands in volume. This expansion can provide a large cooling effect from a fluid escaping through a nozzle such as nozzle 410 of the present disclosure.

In function, the brassiere 200 of the present disclosure can provide mitigating and therapeutic relief prior to, at the outset of, and during a hot flash. At the onset of a hot flash, or during a hot flash, the wearer can use various processes (discussed infra) to exhaust the fluid contained within the fluid source 300 outward through outlet 340 (preferably provided as a plenum 330) into each duct 400 operatively and fluid communicatively connected to outlet 340 toward nozzle 410 and outward therefrom to the upper torso, chest and neck area 520 of the wearer. This easily facilitates the multiple and repeatable action of allowing the wearer to actuate the eHFMS 205 and provide a hot flash mitigating air flow to the upper torso, chest and neck area 520 of the wearer by the repeatable egress of fluid from the fluid source 300 of the eHFMS 205 operably connected to the brassiere 200.

Further, one of skill in the art could provide for a fluid that may also have functional additives provided therein. Such functional additives could include perfumes, medicaments, therapeutic agents, combinations thereof, and the like. Further, one of skill in the art will readily recognize that function additives such as perfumes, medicaments, therapeutic agents, combinations thereof and the like could be directly added to, or provided in cooperation with, the fluid source 300. In other words, a functional additive such as a medicament could be directly added to the internal portion of fluid source 300 by incorporating the functional additive with the fluid taken in by fluid source 300 during routine use. Alternatively, a functional additive such as a medicament would be input directly into the interior of fluid source 300. Yet still, a functional additive such as a perfume could be incorporated into the materials used to form fluid source 300. All of these embodiments should be considered exemplary and non-limiting.

The ducts 400 may be constructed of any material that allows the fluid to be conveyed to the upper torso, chest and neck area 520 of the wearer. This includes but is not limited to plastic or metals. In a preferred embodiment the ducts 400 may be constructed of a soft flexible silicone rubber. The ducts 400 nay have a narrow cross-section. This may be done to make the construction, function, and purpose of the brassiere 200 less obvious to passers-by, resulting in increased emotional comfort of the wearer. Each duct may be directly attached to the material forming each of the two front panel sections 220, interwoven into the material forming each of the two front panel sections 220, or even comprise the material forming each of the two front panel sections 220.

For example, the material used to form a front panel section 220 can comprise discrete hollow fibers (such as capillary fibers). The discrete hollow fibers can be arranged in such a manner that they are provided with a cross sectional size and provided in a distribution that can facilitate the fastest cooling of the upper torso, chest and neck area 520 by facilitating the fastest movement of fluid from the reservoir 300. An exemplary but non-limiting distribution of discrete hollow fibers that can enable an efficient flow of fluid from the reservoir 300 to the upper torso, chest and neck area 520 could be in a fractal pattern similar to natural patterns found in root systems, capillary vessels, and the alveoli in lungs.

Such discrete hollow fibers of the eHFMS 205 can be interwoven into the material forming the two front panel sections 220. This construction can facilitate the connection of each fiber to reservoir 300. It is also possible that each duct 400 be perforated to allow fluid (e.g., air) to escape along the path of the duct 400 to dispense the fluid contained therein at multiple points onto parts of the chest along the path of each duct 400 disposed within or upon the material forming the two front panel sections 220.

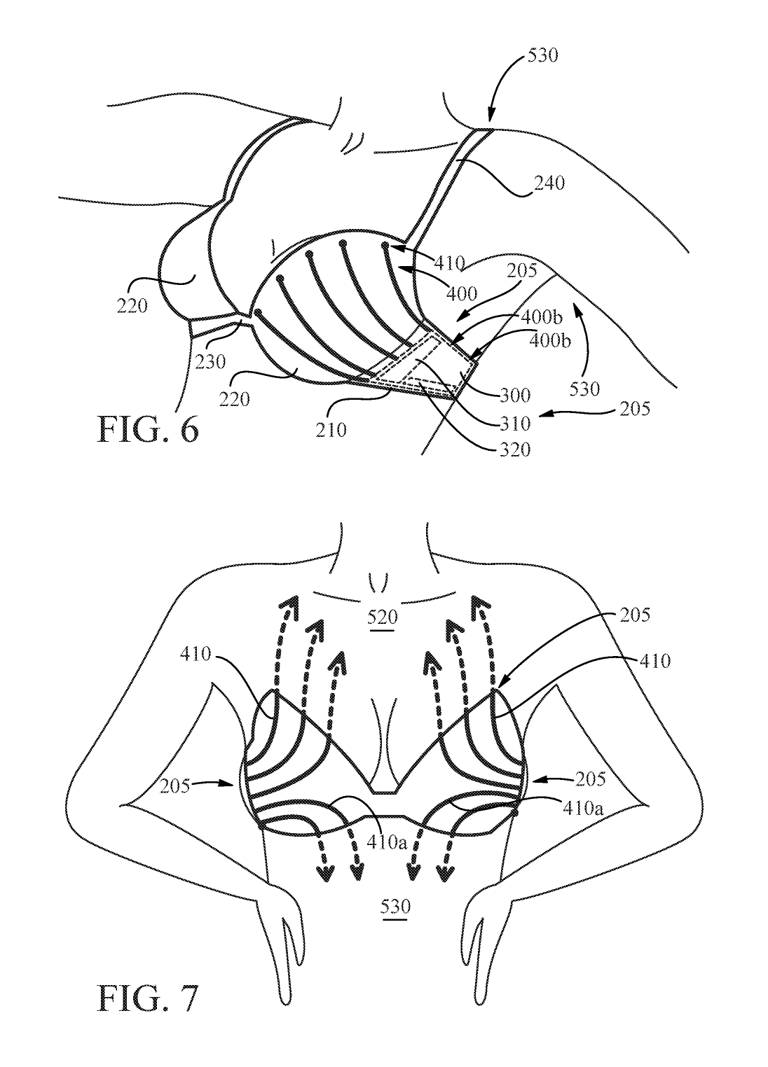

Further, as shown in FIG. 6 the material used to provide the two front panel sections 220 as well as the material used to provide the at least one rear panel section 210 can be provided with cooperatively associated ducts 400, 400B respectively. Here, ducts 400, 400B can direct the flow of fluid outward from each respective nozzle 410 toward the upper torso, chest and neck area 520 of the wearer (i.e., via ducts 400) as well as the upper back and neck 530 of the wearer (i.e., via ducts 400B). In this way, one of skill in the art will recognize that the brassiere 200 and the eHFMS 205 can cool two regions of the upper torso (i.e., the upper torso, chest and neck area 520 and the upper back and neck 530) of the wearer with a single garment when the user experiences a hot flash.

As mentioned supra, a nozzle 410 may be attached to each duct 400 at an end distal from reservoir 300. The nozzles 410 may be used to adjust the flow of air for more air flow or it may be used to restrict airflow, so the fluid doesn't move the wearer's hair.

As shown in FIG. 7, ducts 410A and respective nozzles 410 can be provided within the front panel 220 to direct the flow of fluid outward from each respective nozzle 410 toward the central and/or lower torso 520 (i.e., the region below the breast area) of the wearer. In a similar vein, one of skill in the art could provide both ducts 410 and ducts 410A within or within the respective front panel 220 to provide fluid flow toward both the central and/or lower torso 520 and the upper torso, chest and neck area 520 of the wearer.

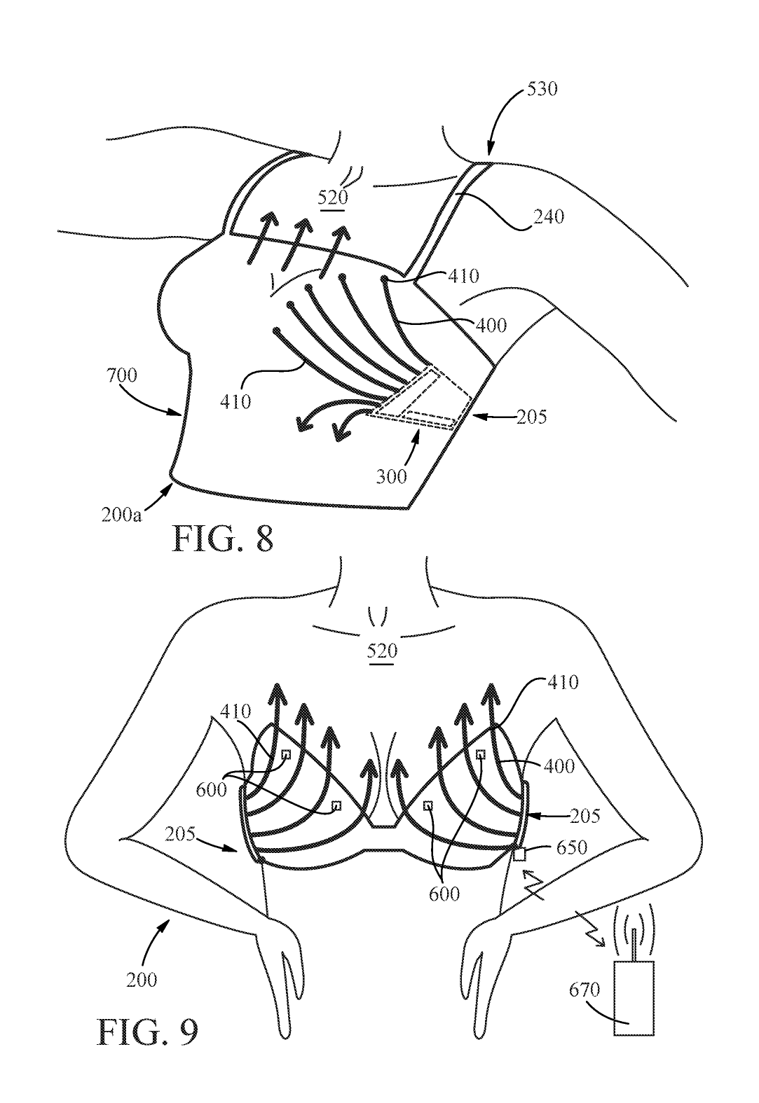

Additionally, as shown in FIG. 8, it should be readily recognized by one of skill in the art that the aforementioned structures (collectively or in part) could easily be incorporated into, and upon the surface of garments 200A other than brassieres 200. By way of non-limiting examples, the reservoir 300 (and the additional structures fluidly and cooperatively associated thereto) as well as the ducts 410 (and any additional structures fluidly and cooperatively associated thereto) could be incorporated into, or upon the surface of, wearable garments such as hats, socks, underwear, shirts, pants, and the like. For example, FIG. 8 provides an exemplary shirt 700 used for wearer workouts that could be provided with any number of reservoirs 300, ducts 400, and nozzles 410 that could facilitate the cooling of the individual as the wearer's work out progresses, the body's core temperature rises, and sweat develops. Or such a shirt could be utilized by a hiker while hiking on a hot summer day to lower the body's core temperature by exhausting air toward regions of the body having major blood vessels.

As shown in FIG. 9, the brassiere 200 and eHFMS 205 can incorporate a wireless body sensor network (or simply BSN) 600. A BSN 600 is a networked collection of a plurality of (programmable) sensor nodes that can communicate among themselves, with other smart devices 670 (discussed supra and infra), and other ambient sensors. The sensor nodes can have computation, storage, wireless transmission, and sensing capabilities. Common physiologically sensed signals/data can include body motion, skin temperature, body temperature change gradient, heart rate, skin conductivity, brain and muscle activity, biomarkers, combinations thereof, and the like. In a preferred embodiment, the BSN 600 is cooperatively associated with the physiological symptoms associated with menopause and/or hot flashes. To perform both online and offline analyses of data streams, BSNs 600 can be assisted by cloud computing-based infrastructures providing flexible storage and scalable processing. A wide range of application scenarios is enabled by BSN 600 technologies, even though m-Health applications probably represent the most emblematic and diffused example. Specifically, BSN 600-based systems can be used to directly monitor several vital signs (such as those associated with the aforementioned hot flashes) continuously and non-invasively, as tiny wireless BSN sensors 600 can be placed on material forming the brassiere 200 and in contacting engagement with the skin of the wearer. In other words, the BSN 600 can be integrated with garments 200A or in the materials and/or the fibers forming materials useful for garments 200A such as brassiere 200.

Moreover, BSNs 600 can be strategic enablers for many other application domains such as: eSport, e-Fitness, e-Wellness, and e-Social. Existing BSN 600 research by one of skill in the art can craft an intimately integratable garment assembly (such as for brassiere 200) from several points of view: hardware (e.g., biosensor boards), communications (e.g. efficient MAC-level protocols), distributed software systems (e.g., collaborative smartphone- and/or BSN-based platforms), and novel applications including advanced data processing algorithms. Additionally, BSNs 600 can be cooperatively and matingly coupled to active-wear devices (e.g., a FITbit®, Apple® watch, and the like)

Additionally, BSNs 600 can be connected to cloud computing by embracing the concept of providing computer resources as a third-party service. Resources include storage, networking, and processing. Different cloud implementations offer different variations of available services. Realized benefits can include dynamic access to resources based on usage demands, and third party management of computing resources. Utilization of cloud computing resources can be provided at a platform level and utilized by customers (e.g., a FITbit®, Apple® watch, and the like). While attractive due to management and utilization efficiencies, there can be costs associated with cloud platforms or configuring BSN 600 data to work with clouds. Complexity may range from recompiling an application for a specific platform to substantial code modifications to access and utilize cloud APIs. Applications can run within closed or secure networks, or connected to identifiable and secure hardware, may operate without securing each individual communication or data transaction. Cloud access is generally over the Internet, rather than restricted to internal access, and hardware resources and connections may be fully under third party control.

In any regard to the integration of such BSN 600 with a garment such as brassiere 200 and/or the hot flash mitigation system 205, the onset of a hot flash can be readily detected by a BSN 600 (perhaps even prior to the wearer's sensation of the onset of a hot flash) and notify the wearer of the impending event via a communication link to a smart device 670 such as a smart phone or any other ‘smart’ device such as a computer. Alternatively, one of skill in the art could integrate a BSN 600 with an electromechanical device 650 that initiates the process of ejecting fluid (e.g., air) from the reservoir 300 cooperatively associated with the brassiere 200 through the ducts 400 in fluid communicating engagement thereto through each nozzle 410 cooperatively associated thereto and onto the upper torso, chest and neck area 520 of the wearer.

For example, biological data from the wearer can be continuously sampled by the wireless body sensor network and monitored by the smart device. Upon the occurrence of a biological trigger, the smart device can activate the eHFMS 205 by providing an activation signal to the microcomputer to activate valve 310 as would be understood by one of skill in the art to facilitate the egress of fluid from fluid source 300. The body sensor network can continuously monitor biological data in situ and as stop signal from the smart device can accordingly cause the cessation of fluid from fluid source 300.

Any dimensions and/or values disclosed herein are not to be understood as being strictly limited to the exact numerical values recited. Instead, unless otherwise specified, each such dimension and/or value is intended to mean both the recited dimension and/or value and a functionally equivalent range surrounding that dimension and/or value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A brassiere 200 incorporating a hot flash mitigation system for cooling the upper torso, chest and neck area 520 of a wearer, said brassiere 200 incorporating a hot flash mitigation system comprising:
a) a brassiere comprising two front panel sections 220 connectively engaged to each other and at least one rear panel section 210 connectively engaged to each of said two front panel sections 220, said connectively engaged two front panel sections and said at least one rear panel section 210 forming a wrap capable of encircling the wearer or intended wearer's body; and,
b) at least one electronic hot flash mitigation system 205 comprising:
1) at least one fluid source 300 having a valve 310 in fluid engagement with an outlet thereof, said valve having a controller cooperatively associated thereto, said controller operating and motivating said valve from a first position wherein a fluid contained in said fluid source 300 cannot egress from said fluid source to a second position wherein said fluid contained within said fluid source 300 can egress from said fluid source 300 to an outlet 340 in cooperative fluid engagement thereto;
2) at least one duct 400 disposed proximate to one of said at least two front panel sections 220 and in fluid engagement with said outlet 340 of said fluid source 300; and,
3) at least one nozzle 410 disposed upon an end of said duct 400 distal from said fluid source 300 and in fluid communication with said at least one duct 400, said at least one nozzle 410 operatively conveying said fluid from said duct to said upper torso, chest and neck area 520 of said wearer.

2. The brassiere 200 of claim 1 wherein said two front panel sections 220 and said at least one rear panel section 210 connectively engaged thereto collectively form a top hole 260, said top hole 260 providing for placement of said brassiere 200 over the head and shoulders 270 of said wearer.

3. The brassiere 200 of claim 1 further comprising at least one shoulder strap 240, said should strap being connectively engaged to one of said two front panel sections 220 and said at least one rear panel section 210 and an arm hole 250 for the insertion of arms 280 of the wearer therethrough.

4. The brassiere 200 of claim 1 wherein said fluid source 300 is a pressurized fluid containing vessel.

5. The brassiere 200 of claim 1 wherein said controller 320 is a mechanical switch.

6. The brassiere 200 of claim 1 wherein said controller 320 further comprises a microcontroller, said microcontroller operating and motivating said valve from said first position to said second position.

7. The brassiere 200 of claim 6 wherein said microcontroller is in communicating engagement with a smart device, said smart device providing an activation signal to said microcontroller to operate and motivate said valve 310 from said first position to said second position.

8. The brassiere 200 of claim 7 wherein said microcontroller is in communicating engagement with a smart device, said smart device providing an activation signal to said microcontroller to operate and motivate said valve 310 from said second position to said first position.

9. The brassiere 200 of claim 1 wherein said outlet 340 further comprises a plenum 330, said plenum 330 being in fluid communication with said fluid source 300 and said duct 400.

10. The brassiere 200 of claim 9 further comprising at least two ducts, each of said at least two ducts 400 being disposed proximate to one of said at least two front panel sections 220 and in fluid engagement with said plenum 330.

11. The brassiere 200 of claim 10 wherein each of said at least two ducts 400 having at least one nozzle 410 disposed upon an end distal from said fluid source 300.

12. The brassiere 200 of claim 1 wherein said one of said two front panel sections 220 is formed from a material, said duct 400 being contactingly and matingly engaged to said material.

13. The brassiere 200 of claim 1 wherein said one of said two front panel sections 220 is formed from a material, said duct 400 being incorporated within said material.

14. The brassiere 200 of claim 1 wherein said duct 400 is a discrete hollow fiber.

15. The brassiere 200 of claim 14 wherein said discrete hollow fiber is disposed within said material forming said one of said two front panel sections 220.

16. The brassiere 200 of claim 1 further comprising at least one wireless body sensor network (BSN) 600 cooperatively and matingly associated thereto, said at least one BSN 600 sensing physiological data of said wearer and communicating said physiological data of said wearer to a smart device.

17. The brassiere 200 of claim 16 wherein said smart device provides an activation signal to said microcontroller to operate and motivate said valve 310 from said first position to said second position when said BSN 600 communicates physiological data of said wearer indicating said wearer is experiencing a hot flash.

18. The brassiere 200 of claim 17 wherein said smart device provides an activation signal to said microcontroller to operate and motivate said valve 310 from said second position to said first position when said BSN 600 communicates physiological data of said wearer indicating said wearer has concluded a hot flash.

19. The brassiere 200 of claim 16 wherein said physiological data of said wearer is selected from the group consisting of body motion, skin temperature, heart rate, skin conductivity, brain and muscle activity, biomarkers, and combinations thereof

20. The brassiere 200 of claim 1 further comprising at least two electronic hot flash mitigation systems 205.
